(19) United States
(12) Patent Application Publication
(10) Pub. No.: US 2022/0076842 A1
(43) Pub. Date: Mar. 10, 2022

(54) MEDICAL INFORMATION PROCESSING SYSTEM AND METHOD

(71) Applicant: Canon Medical Systems Corporation, Otawara-shi (JP)

(72) Inventor: Shigeharu OHYU, Yaita (JP)

(73) Assignee: Canon Medical Systems Corporation, Otawara-shi (JP)

(21) Appl. No.: 17/466,304

(22) Filed: Sep. 3, 2021

(30) Foreign Application Priority Data
Sep. 4, 2020 (JP) 2020-149095
Sep. 2, 2021 (JP) 2021-143128

(51) Int. Cl. G16H 50/30; G16H 50/70; G16H 50/20; G16H 10/60; G06K 9/62

(52) U.S. Cl. CPC G16H 50/30; G16H 50/70; G06K 9/6218; G16H 10/60; G16H 50/20

(57) ABSTRACT

According to one embodiment, a medical information processing system includes a storage and processing circuitry. The storage is configured to store a determination model that determines a probability for a disease. The processing circuitry is configured to: receive first subject information based on a history of a first subject; output first information including the probability based on the determination model and the first subject information; acquire second subject information based on a history of a second subject; update the determination model based on the second subject information; and output second information including the probability of the first subject based on an updated determination model and the first subject information.

### CROSS-REFERENCE TO RELATED APPLICATIONS

This application is based upon and claims the benefit of priority from Japanese Patent Application No. 2020-149095, filed Sep. 4, 2020; and No. 2021-143128, filed Sep. 2, 2021; the entire contents of all of which are incorporated herein by reference.

### FIELD

Embodiments described herein relate generally to a medical information processing system and method.

### BACKGROUND

There is known a medical information processing system that determine a probability of a specific disease for a subject by integrating various test results and information on diagnostic results of a large number of other subjects. For example, diagnoses of the subject' disease, treatment methods, prognoses, risks, etc. are determined.

As such a medical information processing system, for example, there is a technique for estimating a future risk of acquiring a disease from a result of a medical examination, a gene, an age, and a disease history. In this technique, when estimating the risk of acquiring a disease, a risk factor that is difficult to be examined frequently is estimated from easily measurable. items. As a basic technique thereof, there is a method of clustering a distribution of a group to determine a distribution of diseases beforehand as a prior probability distribution, and estimating a posterior probability distribution under actual test results using Bayes' theorem. In this method, the prior probability distribution is estimated based on factor data in the group of subjects, and a posterior probability under a condition where the test results are obtained is determined based on the prior probability distribution. In the correction of the factor data, a factor with smaller variability over a long period of time is input, and a prior distribution of susceptibility is obtained based on this factor. This is because when collecting the factor data in the group of subjects to determine the prior distribution of susceptibility, if the susceptibility of each factor changes during the period in which the data of the group is collected, it becomes impossible to estimate the susceptibility with a high reliability.

On the other hand, in epidemic diseases, a risk of acquiring a disease may fluctuate significantly within a few days depending on the season and situation. In some cases, a behavior of the subject is involved in determining the presence/absence of a disease and determining a treatment after a test. The behavior of the subject can be investigated and traced in interview at the time of the test. However, among the behaviors investigated, those with a high risk may not be known on the day of the test and may be found several days after the test.

For example, suppose that in an initial test, detection of O-157 is conducted by a rapid antigen test, and it is determined to be barely negative, and in a recent dietary inquiry, it is heard in the interview that a salad containing radish sprouts was eaten a few days ago. Here, consider a case where radish sprouts in a specific facility emerges as a source of O-157 infection several days after the test. Considering the general epidemic diseases, in such a case, depending on a possible illness, it may be necessary to contact and ask the subject to come back for a retest. In such a case, a doctor in charge will generally contact the subject to encourage the retest. However, if the number of people tested is large or if information on local risk behaviors such as food intake is not widely informed, the doctor in charge may be unable to determine a necessary treatment and prevent the patient's disease progression promptly.

In this way, when the risk of disease varies significantly in a short period of time, the management of the subject cannot be changed rapidly, and an appropriate treatment may not be taken for the patient.

### BRIEF DESCRIPTION OF THE DRAWINGS

FIG. 1 is a diagram showing an example of a configuration of a medical information processing system according to a first embodiment.

FIG. 2 is a diagram showing a data flow in the medical information processing system according to the first embodiment.

FIG. 3 is a diagram showing an example of a medical interview sheet used for the medical information processing system according to the first embodiment.

FIG. 4 is a diagram showing an example of a record used for the medical information processing system according to the first embodiment.

FIG. 5 is a flowchart exemplifying a processing procedure of medical determination support processing performed by the medical information processing system according to the first embodiment.

FIG. 6 is a diagram showing an example of a data management screen output by the medical determination support processing performed by the medical information processing system according to the first embodiment.

FIG. 7 is a flowchart exemplifying a processing procedure of determination processing performed by the medical information processing system according to the first embodiment.

FIG. 8 is a diagram showing an example of a configuration of a medical information processing system according to a second embodiment.

FIG. 9 is a flowchart exemplifying a processing procedure of determination processing performed by a medical information processing system according to a modification of the second embodiment.

FIG. 10 is a diagram showing an example of a data management screen output by medical determination support processing by a medical information processing system according to a third embodiment.

### DETAILED DESCRIPTION

In general, according to one embodiment, a medical information processing system includes a storage and processing circuitry. The storage is configured to store a determination model that determines a probability for a specific disease. The processing circuitry is configured to: receive first subject information based on a history of a first subject; output first information including the probability of the first subject based on the determination model and the first subject information; acquire second subject information based on a history of a second subject; update the determination model based on the second subject information; and output second information including the probability of the first subject based on an updated determination model and the first subject information.

Hereinafter, embodiments of a medical information processing apparatus will be described in detail with reference to the drawings. In the following descriptions, components having approximately the same function and configuration are denoted by the same reference numerals, and duplicate explanations will be given only where necessary.

### First Embodiment

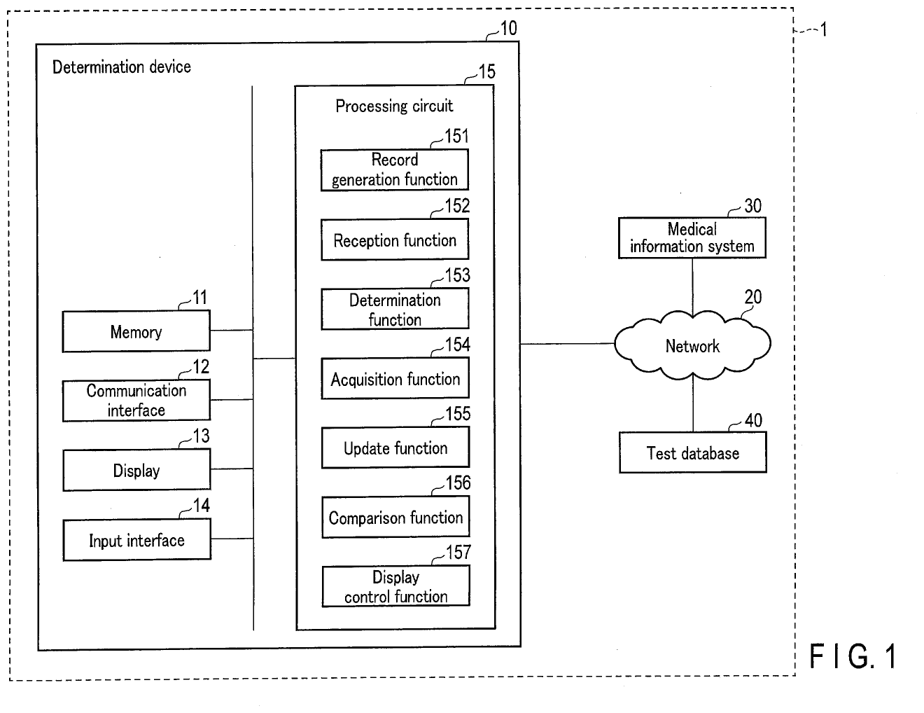

FIG. 1 is a diagram showing a configuration of a medical information processing system 1 of the present embodiment. The medical information processing system 1 includes a determination device 10. The determination device 10 is connected to a medical information system 30 and test database 40 via a network 20. Here, the determination device 10, the medical information system 30, and the test database 40 will be described as being provided in the same facility, but the determination device 10 may be provided in a facility different from that of the medical information system 30 and the examination database 40.

The network 20 is, for example, a LAN (Local Area Network), and the connection to the network 20 may be a wired connection or a wireless connection. Further, if security is ensured by a VPN (Virtual Private Network), etc., the connected line is not limited to a LAN. The device may be connected to a public communication line such as the Internet.

The medical information system 30 manages information relating to a medical facility such as a hospital. The medical information system 30 is, for example, a hospital information system (HIS). In the medical information system 30, an electronic medical record of a subject, information on various tests, a medical examination result, etc. are recorded in a storage device. In the medical examination result, the name of a suspected disease is recorded as an item of “suspicious diagnosis”.

The test database 40 includes a storage device in which a plurality of records relating to a patient (hereinafter, referred to as a subject) suspected of having a specific disease (hereinafter, referred to as a target disease) are recorded. Each record is generated for each subject. A record is generated, for example, each time the subject receives a test or a medical examination at a medical facility. Thus, a plurality of records may be recorded for one subject. The record records medical information of a subject. The medical information includes, for example, a name, a patient ID, information about a test facility, background factors, a behavior history, a medical interview result, test information, diagnostic information, etc. For example, when the target disease is “pneumonia”, the test database 40 may be called a pneumonia test database.

The determination device 10 can transmit and receive various information between the medical information system 30 and the test database 40 via the network 20. The determination device 10 acquires information (hereinafter, referred to as history information) relating to a history of a subject (hereinafter, referred to as a determination subject) to be determined, determines information relating to a probability of a specific disease for the determination subject based on the acquired history information, and outputs a determination result. In the present embodiment, a behavior history or a medical interview result of the determination subject is used as the history information. Further, in the present embodiment, the information relating to a probability of a specific disease for the determination subject is referred to as risk information.

The target disease is, for example, pneumonia. The target disease may be various diseases such as food poisoning due to Escherichia coli and Creutzfeldt-Jakob disease caused by ingesting a specific dangerous part of beef during a specific period.

The risk information is, for example, information including a health condition, the presence/absence of a target disease, and the degree of probability of having contracted the target disease. The risk information may include selection of a treatment method, a prognosis prediction, a prediction of a future risk of acquiring a disease (risk of onset), a prediction of medicine effect, etc. When the risk information includes the degree of probability of having contracted the target disease, the determination device 10 estimates a probability (hereinafter, referred to as a contraction probability) that the determination subject for the target disease has the target disease based on a test result and a behavior history of the determination subject, and based on the estimated contraction probability, determines whether the determination subject corresponds to a “possibility of disease”, a “low probability of disease”, or a “high probability of disease”. For example, a “low probability of disease” indicates that a probability of disease is smaller than a predetermined value. A “high probability of disease” indicates, for example, that a probability of disease is equal to or greater than a predetermined value. A “possibility of disease” indicates, for example, that the magnitude of probability of disease is unknown. The contraction probability may also be referred to as a disease probability.

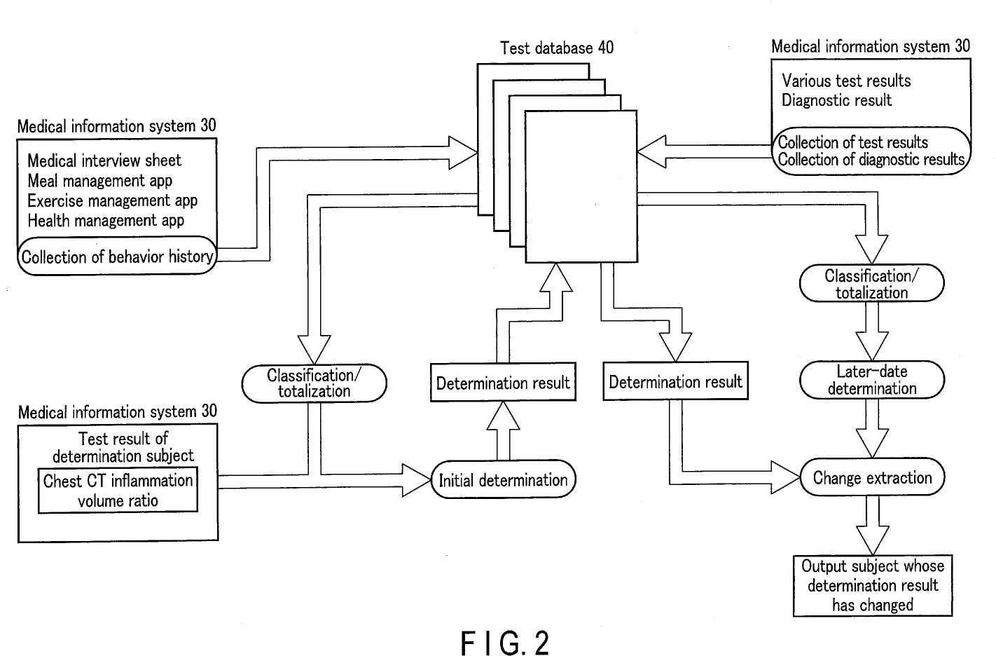

Hereinafter, in the present embodiment, an example will be described in which the target disease is “pneumonia” and the degree of probability that the determination subject has pneumonia is determined as risk information. FIG. 2 is a schematic diagram showing a data flow in the medical information processing system 1 of the present embodiment.

The determination device 10 includes a memory 11, a communication interface 12, a display 13, an input interface 14, and processing circuitry 15. Hereinafter, the determination device 10 will be described as executing a plurality of functions by a single device, but a plurality of functions may be executed by different devices. For example, the functions executed by the determination device 10 may be distributed and mounted in different console devices or workstation devices.

The memory 11 is a storage device such as an HDD (Hard Disk Drive), an SSD (Solid State Drive), or an integrated circuit that stores various information. In addition to the HDD, SSD, etc., the memory 11 may be a portable storage medium such as a CD (Compact Disc), a DVD (Digital Versatile Disc), or a flash memory. The memory 11 may be a drive device that reads and writes various information to and from a semiconductor memory element, etc. such as a flash memory or a RAM (Random Access Memory). Further, a storage area of the memory 11 may be in the determination device 10 or in an external storage device connected by a network.

The memory 11 stores a determination model for at least one disease. Further, the memory 11 stores a program executed by the processing circuitry 15, various data used for processing of the processing circuitry 15, etc. As the program, for example, a program that is installed in a computer from a network or a non-transitory computer-readable storage medium in advance and causes the computer to realize each function of the processing circuitry 15 is used. The various data handled in the present specification are typically digital data. The memory 11 is an example of a storage.

The determination model estimates a contraction probability of a determination subject based on a behavior history or a medical interview result of the determination subject, and determines a probability that the determination subject has a target disease. A publicly known classification algorithm such as linear discriminant analysis is used as the determination model.

The communication interface 12 is a network interface that controls transmission of communication with the medical information system 30 and other external devices via the network 20.

The display 13 displays various information. For example, the display 13 outputs medical information generated by the processing circuitry 15, a GUI (Graphical User Interface) for receiving various operations from an operator, etc. For example, the display 13 is a liquid crystal display or a CRT (Cathode Ray Tube) display. In addition, the display 13 displays a data management screen, etc. to be described later. The display 13 is an example of a display.

The input interface 14 receives various input operations from an operator, converts the received input operations into electric signals, and outputs them to the processing circuitry 15. For example, the input interface 14 receives input of medical information, input of various command signals, etc. from the operator. The input interface 14 is realized by a mouse, a keyboard, a trackball, a switch button, a touch screen in which a display screen and a touch pad are integrated, a non-contact input circuit using an optical sensor, an audio input circuit, etc. for performing various processing, etc. of the processing circuitry 15. The input interface 14 is connected to the processing circuitry 15, and converts the input operations received from the operator into electric signals and outputs them to the control circuit. In the present specification, the input interface is not limited to the one provided with physical operating units such as a mouse and a keyboard. For example, the input interface includes an electric signal processing circuitry that receives an electric signal corresponding to an input operation from an external input device provided separately from the device and outputs this electric signal to the processing circuitry 15. The input interface 14 is an example of an input unit.

The processing circuitry 15 controls operations of the entire determination device 10. The processing circuitry 15 is a processor that executes a record generation function 151, a reception function 152, a determination function 153, an acquisition function 154, an update function 155, a comparison function 156, and a display control function 157 by calling and executing programs in the memory 11.

In FIG. 1, it is assumed that the record generation function 151, reception function 152, determination function 153, acquisition function 154, update function 155, comparison function 156, and display control function 157 are realized by single processing circuitry 15, but processing circuitry may be formed by combining a plurality of independent processors and each processor may execute a program to realize each function. Further, the record generation function 151, the reception function 152, the determination function 153, the acquisition function 154, the update function 155, the comparison function 156, and the display control function 157 may be referred to as a record generation circuit, a reception circuit, a determination circuit, an acquisition circuit, an update circuit, and a display control circuit, respectively, and may be implemented as individual hardware circuits. The above description of each function executed by the processing circuitry 15 also applies to each of the following embodiments and modifications.

The word “processor” used in the above description means, for example, a CPU (Central Processing Unit), a GPU (Graphics Processing Unit), or a circuit such as an ASIC, a programmable logic device (e.g., a Simple Programmable Logic Device (SPLD)), a complex programmable logic device (CPLD), and a field programmable gate array (FPGA). The processor realizes a function by reading and executing a program stored in the memory 11. Instead of storing a program in the memory 11, the program may be directly incorporated in a circuit of the processor. In this case, the processor realizes a function by reading and executing the program incorporated in the circuit. It should be noted that each processor of the present embodiment is not limited to the case where each processor is formed as a single circuit, and a plurality of independent circuits may be combined to form one processor to realize its function. Furthermore, a plurality of constituent elements shown in FIG. 1 may be integrated into a processor to realize its function. The above description of the “processor” also applies to each of the following embodiments and modifications.

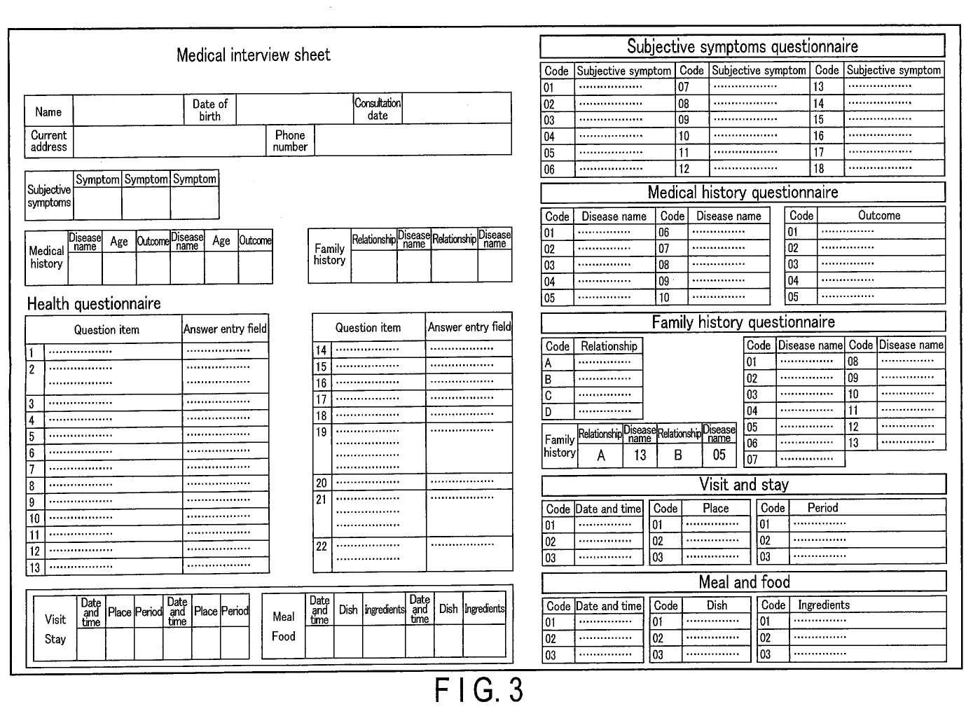

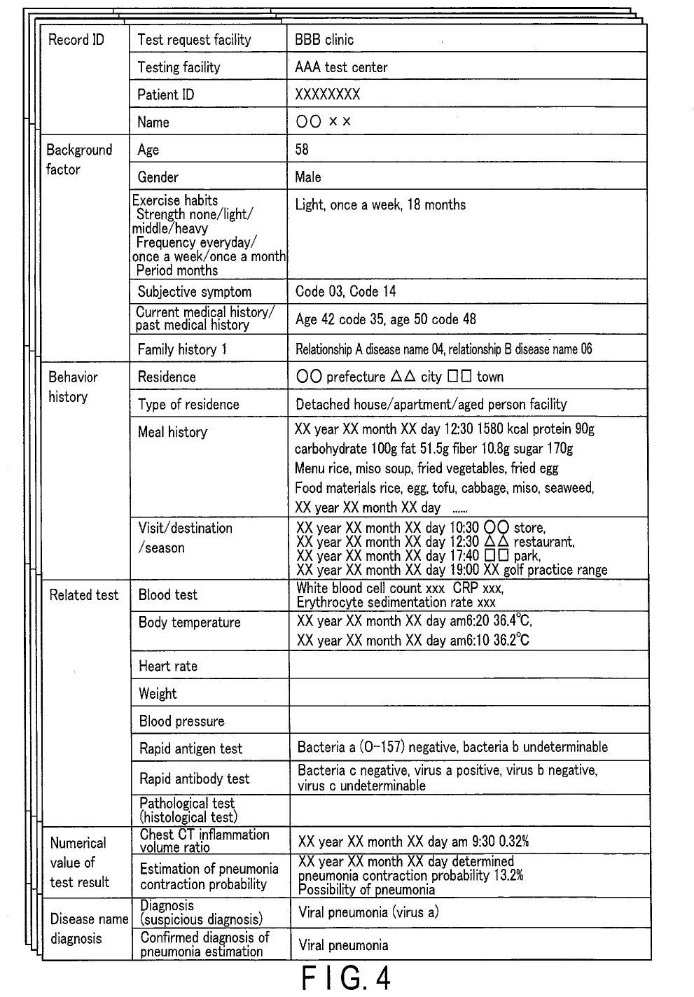

The processing circuitry 15 acquires medical information relating to a subject, and generates a record of the subject based on the acquired medical information, by the record generation function 151. The generated record is, for example, output to the test database 40. The processing circuitry 15 that realizes the record generation function 151 is an example of a report generation unit. The medical information includes a name, a patient ID, information about a test facility, background factors, a behavior history, test information, diagnostic information, a determination result, etc. The medical information is, for example, acquired from the medical information system 30. Alternatively, the medical information is acquired by the subject marking or filling in a medical interview sheet printed on paper and scanning the completed medical interview sheet. The medical information may be acquired from a medical interview result acquired for a meal management application, an exercise management application, a health management application, etc. FIG. 3 is a diagram showing an example of a medical interview sheet used to acquire a behavior history and a medical interview result of a subject. FIG. 4 is a diagram showing an example of a generated record.

Background factors are information relating to factor items with small temporal changes in prior probability regarding parent population. The background factors include, for example, the subject's age, gender, health condition, medical history, and family medical history. The health condition includes, for example, information relating to an exercise habit, a living environment, smoking/drinking, and a subjective symptom.

The behavior history is information relating to a behavior (hereinafter, referred to as a risk behavior) that affects a risk of acquiring a target disease. The behavior history is information relating to factor items with large temporal changes in prior probability. The behavior history includes a place of residence, a type of residence, a meal history, a visit history, a stay history, etc. The meal history includes a type of meal taken, ingredients used in the meal, a date and time when the meal was taken, a name of a product ingested, a date and time when the product was ingested, etc. The visit history includes a name of a facility visited, a date and time of the visit, etc. The stay history includes a name of an area stayed, a season of the stay, a length of the stay, etc. In the medical interview sheet, for example, in each item of the behavior history, a code indicating the content of a risk behavior and a behavior date and time of the risk behavior are input. The behavior date and time includes a date and time when the risk behavior was taken. In this case, a code table is attached to the medical interview sheet. Alternatively, the medical interview sheet contains, for example, an item asking whether or not a specific behavior has been taken during a predetermined period.

The test information is information relating to results of various tests used by a doctor to examine and diagnose the subject. The test information is, for example, acquired from the medical information system 30. The test information includes a test name, a test result, a test date and time, etc. For example, as the test result, a body temperature, a heart rate, a body weight, a blood pressure, etc. are acquired together with a measurement date and time. In addition, for example, as a test result of a blood test, a large number of items such as a white blood cell count and a CPR (C-peptide) value are acquired. Further, for example, as a test result of a rapid antigen test/rapid antibody test, a detection result of a bacterium such as O-157 or another virus is acquired. In addition, as test results of an image test by CT (Computed Tomography), MRI (Magnetic Resonance Imaging), ultrasonic diagnostic equipment, etc., diagnostic imaging findings, and information of measurement values such as a cardiac output, a left ventricular ejection fraction, and a volume ratio of pneumonia findings for an entire lung are acquired.

The diagnostic information includes information relating to an estimation result regarding a probability that the determination subject has a target disease. The diagnostic information is, for example, acquired from the medical information system 30. The diagnostic information is, for example, information indicating “diagnosed as pneumonia”, “diagnosed not as pneumonia”, “no determination as to presence/absence of pneumonia”, etc. Diagnosis is not determined by a result of one test, but is comprehensively determined based on multiple tests, patient background, and medical interview findings. Thus, a confirmed diagnosis is made several days to several weeks after an initial diagnosis. A result (hereinafter, referred to as an initial diagnosis result) of a diagnosis once determined at the time of the initial diagnosis is, for example, recorded as a disease name or a natural language in the item of “suspicious diagnosis” of the medical information system 30. In addition, the initial diagnosis result is recorded in the item of “diagnosis” of the record. A result of the confirmed diagnosis is, for example, recorded as a disease name or a natural language in the item of “confirmed diagnosis” of the medical information system 30. In addition, the result of the confirmed diagnosis is, for example, recorded in the item of “confirmed diagnosis of pneumonia estimation” in the record. The initial diagnosis result may be referred to as a diagnostic finding.

If the result of the confirmed diagnosis is not recorded in the medical information system 30, the processing circuitry 15 may decide a confirmed diagnosis based on the medical information of the subject. As an example, a case will be described where a confirmed diagnosis result of pneumonia is decided based on a pathological test result, a rapid antibody test result, and a disease name of the initial diagnosis result. First, when the presence/absence of pneumonia is determined as a pathological test result, the processing circuitry 15 decides the pathological test result as a confirmed diagnosis result of pneumonia. If the presence/absence of pneumonia cannot be determined as a determination result of the pathological test, or if the pathological test is not performed, the processing circuitry 15 decides a confirmed diagnosis result of pneumonia based on a rapid antibody test result and the disease name of the initial diagnosis result. At this time, if a virus “a” is detected as the rapid antibody test result, the processing circuitry 15 determines the confirmed diagnosis result of pneumonia as “with pneumonia”. When a virus “b” is detected as the rapid antibody test result and a disease name of a provisional diagnosis is “pneumonia” of some kind, the processing circuitry 15 determines the confirmed diagnosis result of pneumonia as “with pneumonia”. If a result other than the above is obtained as the rapid antibody test result, the processing circuitry 15 does not decide the confirmed diagnosis result of pneumonia.

The determination result includes a determination result using a determination model. The determination result is recorded in the item of “estimation of contraction probability of pneumonia” in the record. The record records, for example, “possibility of pneumonia” and “low probability of pneumonia”. For example, if the determination subject has not been determined so far, a determination result is not recorded in the item of “estimation of contraction probability of pneumonia”. In addition, when the determination using a determination model is performed multiple times, a determination result and the date and time when the determination is performed are recorded in the item of “estimation of contraction probability of pneumonia” for each of the multiple times of determinations.

The processing circuitry 15 receives information relating to the determination subject by the reception function 152. Specifically, the processing circuitry 15 extracts a record of a subject for whom a confirmed diagnosis for a target disease is not recorded from all the records stored in the memory 11, and acquires information recorded in the extracted record as information relating to the determination subject. The determination subject is an example of a first subject. The information relating to the determination subject is an example of first subject information. The processing circuitry 15 that realizes the reception function 152 is an example of a reception unit.

In addition, the processing circuitry 15 acquires an attribute value based on the behavior history of the determination subject. The attribute value is a variable set based on each of various behavior items, various test results, a health condition value, etc. Each subject is represented by multidimensional data in which attribute values (variables) for the number of behavior items and attribute values (variables) for the number of other items are combined. Some of these dimensions have discrete values. For example, since the behavior item is a date and time value, the behavior item is treated as a continuous value. Also, for example, the gender item is treated as a discrete value.

The processing circuitry 15 outputs a determination result regarding estimation of a contraction probability of a target disease based on the determination model and the information relating to the determination subject by the determination function 153. The processing circuitry 15 outputs the determination result obtained from the determination model to the test database 40, the display 13, a printing device connected to the determination device 10, etc. The determination result is an example of first information. The determination result may be referred to as first risk information. The processing circuitry 15 that realizes the determination function 153 is an example of an output unit.

For example, the processing circuitry 15 determines one of two clusters, a “low probability of disease” cluster and a “possibility of disease” cluster, the attribute value of the determination subject is belonging in the range of attributes corresponding with the cluster. The “low probability of disease” cluster is a group of subjects whose disease probability of pneumonia is smaller than a predetermined value. The “possibility of disease” cluster is a group of subjects whose disease probability of pneumonia is equal to or greater than a predetermined value.

For example, when a value of a variable of the behavior item or a value of another item variable is included in the range of the “possibility of disease” cluster, the processing circuitry 15 determines “possibility of disease”. In this case, for example, “possibility of pneumonia” is recorded in the item of “estimation of contraction probability of pneumonia” of the record. Further, when a value of a variable of the behavior item or a value of another item variable is included in the range of the “low probability of disease” cluster, the processing circuitry 15 determines “low probability of disease”. In this case, for example, “low probability of pneumonia” is recorded in the item of “estimation of contraction probability of pneumonia” of the record.

The processing circuitry 15 acquires, by the acquisition function 154, information relating to subjects (hereinafter, referred to as a subject with a confirmed diagnosis) for which a confirmed diagnosis for a target disease is recorded. Specifically, the processing circuitry 15 extracts records of the subjects with confirmed diagnosis from all the records stored in the memory 11, and acquires the information relating to the subjects with confirmed diagnosis from the extracted records. The information relating to the subjects with confirmed diagnosis includes at least one of a behavior history and a medical interview result of the subjects with confirmed diagnosis. In addition, the information relating to the subjects with confirmed diagnosis includes confirmed diagnosis results of the subjects with confirmed diagnosis. The subjects with confirmed diagnosis is an example of a second subject. The information relating to the subjects with confirmed diagnosis is an example of second subject information. The confirmed diagnosis result of the subjects with confirmed diagnosis is an example of diagnostic information of the second subject. The processing circuitry 15 that realizes the acquisition function 154 is an example of an acquisition unit. Further, the processing circuitry 15 acquires an attribute values based on the behavior history of the subjects with confirmed diagnosis.

The processing circuitry 15 updates, by the update function 155, the determination model based on the information relating to the subjects with confirmed diagnosis and the diagnostic information of the subjects with confirmed diagnosis. At this time, the processing circuitry 15 updates the determination model using at least one of the behavior history and the medical interview result of the subjects with confirmed diagnosis and the confirmed diagnosis results of the subjects with confirmed diagnosis. The processing circuitry 15 that realizes the update function 155 is an example of an update unit.

Records of a large number of subjects are newly generated in the test database 40, and various behavior items and health conditions of the large number of subjects are recorded daily. In addition, a subjects with confirmed diagnosis having a confirmed diagnosis are added to the test database 40 daily. The processing circuitry 15 updates, by the update function 155, a condition (hereinafter, referred to as a determination condition) for performing determination regarding estimation of a contraction probability of a target disease by performing classification/totalizing processing to be described later on all the subjects with confirmed diagnosis including the newly added subjects with confirmed diagnosis. By updating the determination condition, the determination model is updated.

Specifically, the processing circuitry 15 first classifies the subjects with confirmed diagnosis into a plurality of clusters based on the behavior items and the behavior date and time of the subjects with confirmed diagnosis. At this time, the processing circuitry 15 calculates a disease probability of each of the plurality of clusters and classifies them so that a difference in disease probabilities between the clusters becomes large.

The processing circuitry 15 outputs a determination result after the update based on an updated determination model and the information relating to the determination subjects by the determination function 153. The processing circuitry 15 performs determination again using the determination model, updates the determination result based on a redetermination result of the determination model, and outputs the determination result after the update to the test database 40, the display 13, a printing device connected to the determination device 10, etc. The determination result after the update is an example of second information. The determination result after the update may be referred to as second risk information.

Specifically, the processing circuitry 15 determines cluster in which one of the plurality of clusters the determination subject is included based on the behavior date and time of the determination subject, and outputs a determination result regarding estimation of a contraction probability based on the disease probability of the cluster.

The processing circuitry 15 compares the determination results before and after the update for each of the determination subjects, and outputs a comparison result regarding a subject whose determination results before and after the update are different, by the comparison function 156. For example, when the determination result has changed, the processing circuitry 15 outputs information indicating that the determination result has changed to the display 13 or a printing device connected via the network 20, etc. At this time, the processing circuitry 15 may create a list in which only the subjects whose determination results have changed are extracted and output the list to the display 13 or the printing device. The processing circuitry 15 that realizes the comparison function 156 is an example of a comparison unit.

The processing circuitry 15 causes the display 13 to display a GUI (hereinafter, referred to as a data management screen) for managing the data stored in the test database 40, by the display control function 157. The processing circuitry 15 that realizes the display control function 157 is an example of a display control unit.

Next, an operation of the medical determination support processing executed by the determination device 10 will be described. The medical determination support processing is processing of collecting behavior histories and confirmed diagnosis results regarding a target disease for a plurality of subjects, estimating a contraction probability of a determination subject for the target disease based on the collected results, performing determination regarding the estimation of the contraction probability, and when a determination result has changed from a previous determination result, outputting information indicating that the determination result has changed.

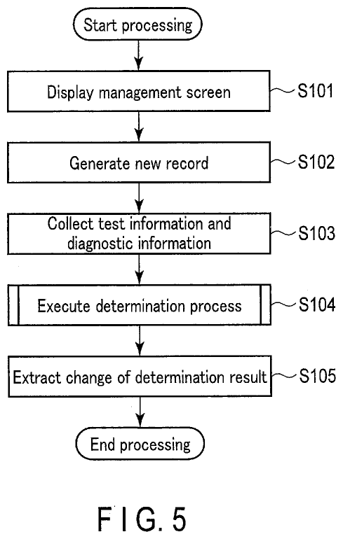

Hereinafter, a case where a process of each step of the medical determination support processing is executed by an instruction being input by a user in the input interface 14 will be described. FIG. 5 is a flowchart showing an example of a procedure of the medical determination support processing. The processing procedure in each processing described below is only an example, and each processing can be changed as appropriate where possible. Further, with respect to the processing procedure described below, steps can be omitted, replaced, and added as appropriate according to the embodiment.

A process of each step of the medical determination support processing may be automatically executed at regular intervals. The medical determination support processing may be executed, for example, once a day at a predetermined time at midnight. In addition, the medical determination support processing may be executed every week (7 days). In addition, the medical determination support processing may be executed each time a record of a new subject is generated. The medical determination support processing may be referred to as batch processing.

(Medical Determination Support Processing)

(Step S101)

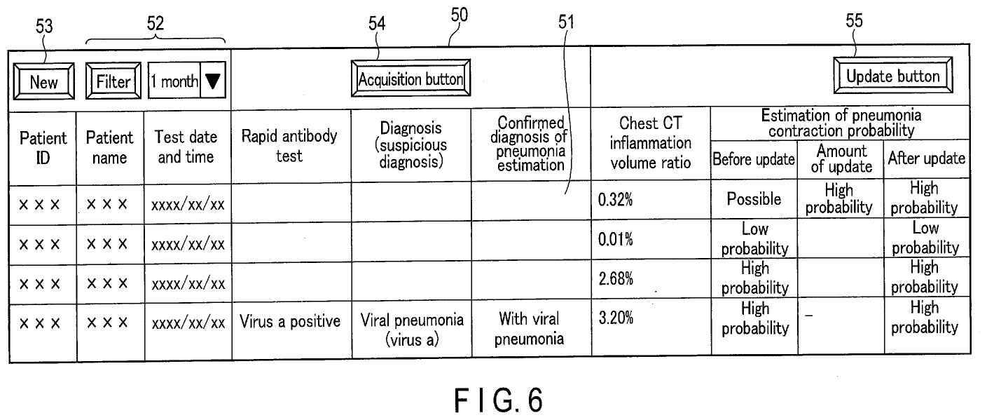

The processing circuitry 15 causes the display 13 to display the data management screen 50 based on a record stored in the memory 11, by the display control function 157. FIG. 6 is a diagram showing an example of the data management screen 50. The data management screen 50 includes a data display part 51, a period setting part 52, a record creation instruction input part 53, a data collection instruction input part 54, and an update instruction input part 55.

Information relating to the record stored in the memory 11 of the determination device 10 for each subject is listed and displayed in the data display part 51. The data display part 51 displays, for example, a name, a patient ID, information about a test facility, background factors, a behavior history, test information, diagnostic information, a determination result, etc. In a display column of the test information, for example, a test result of a rapid antibody test is displayed. In a display column of the diagnostic information, for example, an initial diagnosis result is displayed. The test result of the rapid antibody test and the initial diagnosis result are preferably displayed because they are used for a confirmed diagnosis.

In the period setting part 52, an instruction to set a record to be displayed in the data display part 51 is input. For example, by setting “1 month” in the period setting part 52, only records created in the last one month are displayed in the data display part 51.

In the record creation instruction input part 53, an instruction to newly create a record is input. In the data collection instruction input part 54, an instruction to acquire test information and diagnostic information is input. In the update instruction input part 55, an instruction to update a determination result regarding a target disease of a determination subject is input.

(Step S102)

When an operation is input in the record creation instruction input part 53, the processing circuitry 15 scans a medical interview sheet and generates a new record using the scan data, by the record generation function 151.

(Step S103)

When an operation is input in the data collection instruction input part 54, the processing circuitry 15 acquires electronic medical records of the subjects displayed in the data display part 51 from the medical information system 30, and collects attributes such as test information, behavior history and diagnostic information of subjects for whom “pneumonia” is recorded in the item of “suspicious diagnosis”. As a result, attributes such as test information and diagnostic information that have not been recorded in the record so far are newly acquired. Only extracted records are displayed in the data display part 51. In an example of FIG. 6, among the records created in the last one month, there are four subjects for whom “pneumonia” is recorded in the item of “suspicious diagnosis” of the electronic medical record.

(Step S104)

When an operation is input in the update instruction input part 55, the processing circuitry 15 extracts a subject whose confirmed diagnosis does not exist from the records displayed in the data display part 51 as a determination subject. Then, the processing circuitry 15 acquires a determination result by executing a process (hereinafter, referred to as a determination process) of performing determination regarding estimation of a contraction probability of a target disease by the reception function 152, the determination function 153, the acquisition function 154, and the update function 155. The determination result is stored in the record. Detailed processing of the determination process will be described later.

(Step S105)

The processing circuitry 15 compares determination results of the last two times stored in the record of the determination subject by the comparison function 156. When the determination results are different, the processing circuitry 15 extracts a change in determination result and displays it in the data display part 51. In the example of FIG. 6, in a display column of the determination result of the data display part 51, a determination result before update, a determination result after the update, and a comparison result between the determination result before the update and the determination result after the update are displayed. When only one determination result is stored in the record, a determination result before update is not displayed, and only a determination result after the update is displayed in the data display part 51. When two or more determination results are stored in the record, the data display part 51 displays a previous determination result as a determination result before update, and displays a latest determination result as a determination result after the update. When the previous determination result and the latest determination result are different, the data display part 51 displays a change in determination result or the latest determination result as a comparison result. In the data display part 51, in a determination result for a subject having a confirmed diagnosis, a determination result before update and a latest determination result as a determination result after the update are displayed, and a symbol, etc. indicating that it is not applicable for comparison is displayed in a comparison result.

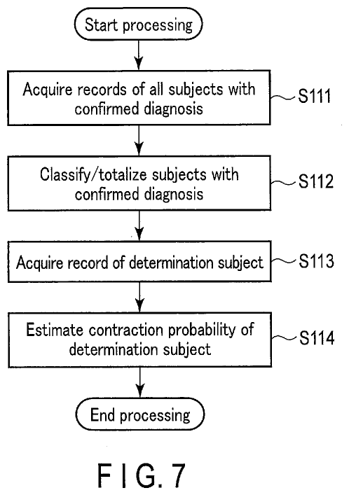

Next, the operation of the determination process executed by the determination device 10 will be described in detail. FIG. 7 is a flowchart showing an example of a procedure of the medical determination support processing.

(Determination Process)

(Step S111)

The processing circuitry 15 refers to the test database 40, and acquires records of all the subjects for whom a confirmed diagnosis of pneumonia has been made as records of subjects with confirmed diagnosis, by the acquisition function 154. Next, the processing circuitry 15 acquires an attribute value of each of the subjects with confirmed diagnosis and a confirmed diagnosis result of each of the subjects with confirmed diagnosis based on the records of the subjects with confirmed diagnosis. At this time, the processing circuitry 15 acquires an attribute value of only a behavior item having a behavior date and time within an expiration date. Here, as a confirmed diagnosis result, either “with disease” or “without disease” is acquired.

(Step S112)

Next, the processing circuitry 15 updates a determination model by the update function 155. At this time, the processing circuitry 15 first applies various classification methods to the attribute value of each subject with confirmed diagnosis, and classifies each subject with confirmed diagnosis into either a “low probability of disease” cluster or a “possibility of disease” cluster. At this time, various variable selection methods may be used to reduce number of variables used for classification. The variable selection methods are, for example, a variable decrease method or a variable increase method. Other variable selection methods may be used. When the number of variables is not huge, it is preferable to use an exhaustion method as the classification method from the viewpoint of accuracy.

Further, the processing circuitry 15 divides an area of attribute values so that a ratio of the number of subjects in a group (hereinafter, referred to as a with-disease group) of subjects whose confirmed diagnosis is “with disease” and the number of subjects in a group (hereinafter, referred to as a without-disease group) of subjects whose confirmed diagnosis is “without disease” is as different as possible between the “low probability of disease” cluster and the “possibility of disease” cluster. That is, the processing circuitry 15 decides a condition (hereinafter, referred to as a classification condition) for classifying subjects with confirmed diagnosis based on the attribute values so that a subject whose confirmed diagnosis is “with disease” is classified into the “possibility of disease” cluster and a subject whose confirmed diagnosis is “without disease” is classified into the “low probability of disease” cluster.

When a linear discriminant analysis is used as the classification method (clustering method), they are divided so that a Mahalanobis distance between the clusters is maximized. Many clustering methods decide a division curved surface based on some distance between two groups. For example, in a support vector machine, a division curved surface is decided so as to maximize a distance between a group and the dividing surface. The distance is a distance in an attribute value space. In addition, as another classification method, there is a classification method using a decision tree. In the classification method using a decision tree, data is classified so as to minimize impurity of the data when the data is divided.

Further, as the classification method (clustering method), a method as described in Japanese Patent Application No. 2020-039545, which is an unpublished prior application, may be used. The prior application describes a method of deciding clusters so that an upper limit value of a confidence interval of a disease probability is minimized or a lower limit value of the confidence interval of the disease probability is maximized in order to classify the disease probabilities between the clusters by separating them as much as possible. This method is efficient in a problem of poorly separated clusters, for example, even if the disease probability of the “possibility of disease” cluster is much lower than 100% (e.g., about 5%), when it can be shown with a sufficient number of data that it is higher than the disease probability (e.g., 0.1%) of the “low probability of disease” cluster, ranges of those clusters can be decided. The range of a cluster includes a date and time range. It is difficult to classify such poorly separated clusters by distance-based or impurity-based dividing methods. Below, as an imaginary example, a classification result will be described of a case where, regarding a behavior item of eating venison raw in Okayama prefecture, there are a large number of subjects whose confirmed diagnosis is “with disease” among subjects whose behavior date and time is during Jan. 10 to 14, 1997 and there are a small number of subjects whose confirmed diagnosis is “without disease” among subjects whose behavior date and time is during a period other than the above. A subject with a rapid antigen test numerical value equal to or greater than a certain value is classified into the “possibility of disease” cluster regardless of the behavior item of eating venison raw in Okayama prefecture. In addition, a subject who, even if a rapid antigen test numerical value is smaller than a certain value, ate venison raw during Jan. 10 to 14, 1997 is classified into the “possibility of disease” cluster. On the other hand, a subject whose rapid antigen test numerical value is smaller than a certain value and who did not eat venison raw during Jan. 10 to 14, 1997 is classified into the “low probability of disease” cluster.

The processing circuitry 15 performs totalizing processing for each cluster based on the classification result of the subjects with confirmed diagnosis, and calculates a disease probability of each cluster based on a totalizing result. The disease probability of each cluster is expressed by the following formula.

Disease probability=number of tests of with-disease group/(number of tests of without-disease group+number of tests of with-disease group)

Here, the number of tests of with-disease is the number of subjects in the with-disease group among the subjects included in this cluster. The number of tests of without disease is the number of subjects in the without-disease group among the subjects included in this cluster. That is, the disease probability is a ratio of the number of subjects in the with-disease group among the subjects included in this cluster to a total number of subjects included in the cluster. The processing circuitry 15 classifies each subject with confirmed diagnosis so that the disease probabilities between the clusters are separated as much as possible. That is, the processing circuitry 15 decides classification criteria of the subject so that a difference in disease probabilities between the clusters becomes as large as possible based on the disease probability of each cluster.

It is preferable that the processing circuitry 15 use only an attribute value of a behavior item within an expiration date for the classification. An expiration date is set for each behavior item. The expiration date is a period during which a behavior item affects determination of a subject. For example, in a behavior item indicating date and time when “shrimp (raw)” is eaten as food, 10 days is set as an expiration date. In this case, the day 10 days before the determination date is set as a valid period date. A behavior item indicating that the shrimp was eaten before the expiration date does not provide effective information for determination of a subject to be determined. Thus, the processing circuitry 15 does not use an attribute value of the behavior item having the behavior date and time before the expiration date for classification, and uses only an attribute value of a behavior item having a behavior date and time in a period from the expiration date to the determination date for classification.

In addition, for a behavior item in which the number of subjects used for classification is a certain number or less, classification may be performed without using the behavior item. For example, for a certain behavior item, when the number of subjects having a behavior date and time within an expiration date is a certain number or less, classification of subjects with confirmed diagnosis is performed without using an attribute value of the behavior item. In this case, by reducing the number of items used for classification, it is possible to prevent the behavior items of the subjects used for classification from reaching a huge number of types, and the classification process can be performed in a practical time.

(Step S113)

The processing circuitry 15 acquires records of determination subjects by the reception function 152. Next, the processing circuitry 15 acquires an attribute value of each of the determination subjects based on the records of the determination subjects.

(Step S114)

The processing circuitry 15 determines, by the determination function 153, in which one of the “low probability of disease” cluster and the “possibility of disease” cluster each determination subject is included based on the attribute value of the determination subject and the classification conditions of the clusters. For a determination subject in which values of a variable of a behavior item and another variable are included in the classification condition of the “possibility of disease” cluster, the processing circuitry 15 sets a disease probability of the “possibility of disease” cluster as a contraction probability of the determination subject and determines “possibility of disease” for an estimation result of a pneumonia contraction probability. For a determination subject in which values of a variable of a behavior item and another variable are included in the condition of the “low probability of disease” cluster, the processing circuitry 15 sets a disease probability of the “low probability of disease” cluster as a contraction probability of the determination subject and determines “low probability of disease” for an estimation result of a pneumonia contraction probability.

As described above, by executing the processes of steps S101 to S105, a determination result in the determination process can be obtained for a subject for whom a determination is performed for the first time. For a subject for whom a second or subsequent determination is performed by the determination process, a latest determination result can be obtained by using a determination model that reflects a diagnostic result of a subject with confirmed diagnosis added from the date of the previous determination to the time of the determination this time. Then, when the determination result this time has changed from the previous determination result, for example, the data management screen 50 displaying a change in determination result is displayed on the display 13. The data management screen 50 may be output to a printing device connected via the network 20, etc., and may be printed on paper by the printing device. Further, a list in which only the subjects whose determination results have changed are extracted may be created and output to the display 13 or the printing device.

Hereinafter, advantageous effects of the medical information processing system 1 having the determination device 10 according to the present embodiment will be described.

Records of a large number of subjects are newly generated in the test database 40, and various behavior items and health conditions of the large number of subjects are recorded daily. In addition, a subject with confirmed diagnosis having a confirmed diagnosis is added to the test database 40 daily. In a determination based on an initial test numerical value, only a risk factor known at that moment is considered. A behavior history of a determination subject is related to determination of presence/absence of a disease and a treatment after a test. In addition, a relationship between a behavior and a risk of acquiring a disease of a subject is sometimes revealed later. Thus, a behavior item that affects a contraction probability may change in a short period of time. For example, it may be found that a person who behaves similarly during a particular period has a similar disease.

The medical information processing system 1 according to the present embodiment receives subject information based on history information of a determination subject, and based on a determination model for a specific disease and the subject information of the determination subject, outputs information relating to a probability of a specific disease for the determination subject. The information relating to a probability of a specific disease may be referred to as risk information. The history information includes at least one of a behavior history and a medical interview result. Further, the medical information processing system 1 acquires subject information based on at least one of a behavior history and a medical interview result of a subject with confirmed diagnosis, and updates a determination model based on the subject information of the subject with confirmed diagnosis. Then, the medical information processing system 1 outputs updated information about the determination subject based on the updated determination model and the subject information of the determination subject.

In the present embodiment, as information relating to a probability of a specific disease, a contraction probability for having contracted a specific disease is used. In addition, the subject information of the subject with confirmed diagnosis includes a confirmed diagnosis of the subject with confirmed diagnosis regarding a specific disease. The medical information processing system 1 updates a determination model based on the confirmed diagnosis of the subject with confirmed diagnosis.

Further, the medical information processing system 1 according to the present embodiment acquires behavior items and a behavior date and time of the subjects with confirmed diagnosis based on the subject information of the subjects with confirmed diagnosis, and based on the behavior items and the behavior date and time of the subjects with confirmed diagnosis, classifies the subjects with confirmed diagnosis into a plurality of clusters. At this time, the processing circuitry 15 calculates a disease probability of each of the plurality of clusters and classifies them so that a difference in disease probability between the plurality of clusters becomes large. Then, the medical information processing system 1 determines in which one of the plurality of clusters a determination subject is included based on a behavior date and time of the determination subject, and outputs a determination result regarding estimation of a contraction probability based on the disease probability of that cluster.

With the above configuration, according to the medical information processing system 1 according to the present embodiment, a determination model is updated using behavior items and a behavior date and time of all subjects with confirmed diagnosis including a subject for whom a confirmed diagnosis is newly added. As a result, a diagnostic result of a subject for whom a confirmed diagnosis is made from the day when the previous determination was made to the time when the determination this time is made is reflected in the determination model. Then, by determining a risk of acquiring a disease for a disease using the updated determination model, it is possible to obtain a determination result that reflects a change in epidemics and a change in risk factors in a region. As a result, even if the risk of acquiring a disease changes significantly in a short period of time, a management of a subject can be rapidly changed and an appropriate treatment can be taken for the patient.

Further, the medical information processing system 1 according to the present embodiment compares a determination result after the update and a determination result before the update for each of the determination subjects, and outputs a comparison result for a subject whose determination results before and after the update are different. For example, when the current determination result has changed from the previous determination result, the data management screen 50 displaying the change in determination result is displayed on the display 13. By checking the data management screen 50 on which the change in determination result is displayed, the user can grasp the subject whose risk of acquiring a disease has changed and take appropriate measures.

Further, the data management screen 50 on which the change in determination result is displayed may be output to a printing device connected via the network 20, etc. and printed on paper by the printing device. For example, when the determination process is automatically executed at midnight and a list of subjects whose test results have changed in re-determination at a later date is printed, by checking the list the next day, an attending physician can grasp the subjects whose determination results have changed. For example, if a retest is required, the subject can be contacted to schedule the next test. In addition, when the test result changes for the worse, necessary measures such as a retest can be taken by checking the list of subjects whose determination results have changed. This makes it possible to prevent overlooking a subject who may have a disease. In addition, when a determination result of a subject changes for the better, if the subject shows good progress, it is determined that an actual risk at the time of the previous determination was not large, and a determination to terminate an observation and treatment can be made.

Further, in the present embodiment, the medical information processing system 1 generates a confirmed diagnosis based on a test result and diagnostic findings of a subject with confirmed diagnosis. As a result, even if a result of a confirmed diagnosis is not recorded in the medical information system 30, for example, a confirmed diagnosis can be acquired based on a test result of a pathological test, a test result of a rapid antibody test, and a disease name of the diagnostic findings.

### Second Embodiment

A second embodiment will be described. The present embodiment is obtained by modifying the configuration of the first embodiment as follows. Descriptions of the same configuration, operation, and effect as in the first embodiment will be omitted. The medical information processing system 1 according to the present embodiment uses the results of the classification/totalizing processing in the above-described determination process to extract a behavior item that gives a certain amount or more of influence to a diagnosis.

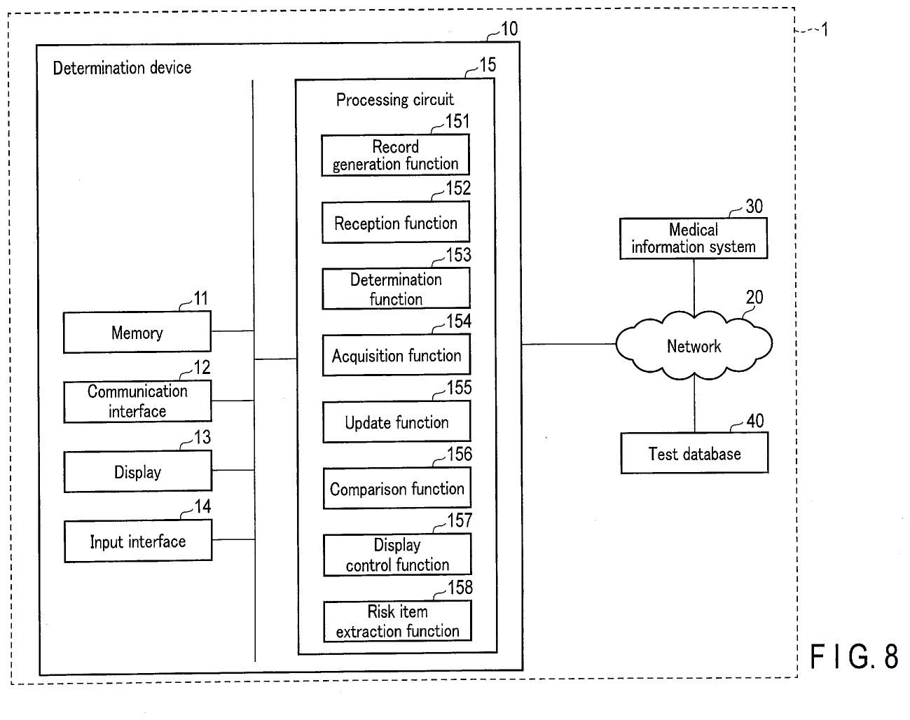

FIG. 8 is a diagram showing a configuration of the medical information processing system 1 of the present embodiment. The processing circuitry 15 executes a risk item extraction function 158 in addition to each function described in the first embodiment. The processing circuitry 15 extracts a high-risk behavior item (hereinafter, referred to as a risk item) based on information relating to a subject with confirmed diagnosis and diagnostic information of the subject with confirmed diagnosis, and outputs the extracted risk item to the test database 40, the display 13, a printing device connected to the determination device 10, etc., by the risk item extraction function 158. The risk item is a behavior item that gives a certain amount or more of influence to a diagnosis. For example, the processing circuitry 15 determines a behavior item included in the “possibility of disease” cluster as the risk item. Processing circuitry that realizes the risk item extraction function 158 is an example of an extraction unit.

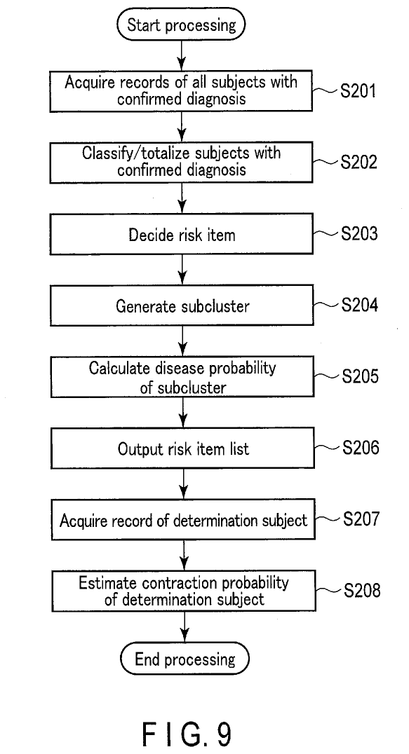

Next, an operation of a determination process executed by the determination device 10 of the present embodiment will be described. FIG. 9 is a flowchart showing an example of a procedure of the determination process according to the present embodiment. Since the processes of steps S201-S202 and S207-S208 are the same as the processes of steps S111-S114 in FIG. 7, respectively, descriptions thereof will be omitted. Here, an example will be described in which each of a plurality of behavior items included in the “possibility of disease” cluster is extracted as a risk item, and the extracted risk item is output to the display 13.

(Determination Process)

(Step S203)

The processing circuitry 15 decides each of the plurality of behavior items included in the “possibility of disease” cluster as a risk item by the risk item extraction function 158.

(Step S204)

The processing circuitry 15 generates a plurality of subclusters by classifying an area of the “possibility of disease” cluster into a plurality of areas centered on each of the behavior items by the risk item extraction function 158. Each of the generated subclusters corresponds to one of the behavior items included in the “possibility of disease” cluster. A range of behavior dates and times of the risk item is also included in a range of a subcluster.

(Step S205)

The processing circuitry 15 applies the above-described totalizing processing to each subcluster, and calculates a disease probability of each subcluster and its confidence interval, by the risk item extraction function 158.

(Step S206)

The processing circuitry 15 generates a list (hereinafter, referred to as a risk item list) of risk items by the risk item extraction function 158. In the risk item list, regarding each of the subclusters, a core risk item, a date and time range, a disease probability, a confidence interval for disease probability, the number of subjects in a with-disease group, and the number of subjects in a without-disease group are described. The processing circuitry 15 outputs the generated risk item list to the display 13.

A facility staff working on the risk item extraction can perform a confirmation operation for each risk item displayed in the risk item list. For example, an “approve” button and a “disapprove” button are displayed for each subcluster on a display screen of the risk item list, and the user can select whether or not to use each risk item for the subsequent determination process by specifying either the “approve” button or the “disapprove” button. In the subsequent determination process, only a risk item corresponding to a subcluster for which the “approve” button is selected is used, and a risk item corresponding to a subcluster for which the “disapprove” button is selected is not used. For example, for a risk item corresponding to a subcluster for which the “disapprove” button is selected, even if a determination subject belongs to that subcluster in the subsequent determination process, it is determined to be “low probability of disease”, not “possibility of disease”.

Hereinafter, advantageous effects of the medical information processing system 1 having the determination device 10 according to the present embodiment will be described.

The medical information processing system 1 according to the present embodiment extracts a risk item based on information relating to a subject with confirmed diagnosis and diagnostic information of the subject with confirmed diagnosis, and outputs the extracted risk item. The risk item is a behavior item that has a certain amount or more of influence on a diagnostic result. For example, a behavior item included in the “possibility of disease” cluster is extracted as a risk item. The risk items are, for example, listed and displayed on the display 13.

With the above configuration, according to the medical information processing system 1 according to the present embodiment, a worker can know a behavior item that gives a certain amount or more of influence to a diagnosis by checking the output risk items. In addition, the worker can discretionarily select a behavior item to be used for determination regarding estimation of a contraction probability from the risk items.

The extracted risk items may be output to a medical interview sheet creation device that creates a medical interview sheet. In this case, the processing circuitry 15 outputs the extracted risk items to the medical interview sheet creation device as question items to be added to the medical interview sheet by the risk item extraction function 158. The medical interview sheet creation device receives the extracted risk items and adds the question items for collecting behavior histories of the risk items to the medical interview sheet. For example, question items regarding epidemic-independent constant risks, such as constant infection risk areas overseas, are always included in the medical interview sheet. Furthermore, by adding a question item regarding a behavior item newly found by the risk item extraction process to the medical interview sheet, it is possible to appropriately collect a behavior history regarding a risk item newly found by a recent diagnostic result.

In the above-described embodiment, the configuration in which the test database 40 is installed in a facility and data is recorded for a subject who undergoes a medical examination at a test facility has been described, but the present invention is not limited thereto. The test database 40 may be constructed to collect regional or national data in order to collect more subject data. The test database 40 may be a distributed database. In such a case, data needs to be anonymized when providing the data outside the facility or when retrieving the data from a regional database. As a method of anonymizing data, for example, k-anonymization, which indistinguishably anonymizes less than k subjects, can be used. For example, it is desirable to apply k-anonymization at k=3. Unless at least a few subjects are included in a cluster, the confidence interval for disease probability becomes wide and a reliable disease probability cannot be calculated. Thus, even if the cluster is limited to not being less than three people, anonymity can be maintained so as not to cause a practical problem.

### Third Embodiment

A third embodiment will be described. The present embodiment is obtained by modifying the configuration of the first embodiment as follows. Descriptions of the same configuration, operation, and effect as in the first embodiment will be omitted.

The determination device 10 estimates an effect of a specific medicine (hereinafter, referred to as a medicine effect) for a determination subject based on a behavior history of the determination subject, and based on the estimated medicine effect, determines whether the determination subject corresponds to “possibility of disease improvement”, “low probability of disease improvement”, or “high probability of disease improvement”. For example, the “low probability of disease improvement” indicates that a probability that a disease will improve by administering a medicine is smaller than a predetermined value. The “high probability of disease improvement” indicates, for example, that a probability that a disease will improve by administering a medicine is equal to or greater than a predetermined value. The “possibility of disease improvement” indicates, for example, that a magnitude of a probability that a disease will improve by administering a medicine is unknown. In the present embodiment, test information and information relating to a medicine (hereinafter, referred to as medicine information) are used as the history information. The medicine information is information relating to a medicine used for treatment of the determination subject. The medicine information includes a type, a name, an administration start date, administration date and time, a dose, etc. of a medicine used for treatment.

The test information is, for example, a test result of a simple test such as a rapid antigen test or a rapid antibody test. In this case, the test result is a detection result of bacteria or viruses.

A determination model determines a probability that a condition of a determination subject to whom a specific medicine has been administered will improve. Specifically, the determination model estimates a medicine effect of a specific medicine for a determination subject based on a behavior history of the determination subject, and determines a probability that a disease of the determination subject will improve.

In addition, the determination device 10 collects confirmed diagnosis results regarding medicine effects for a plurality of subjects, estimates a medicine effect of a specific medicine for a specific subject based on the collected results, and determines a probability that the disease of the determination subject will improve. When the determination result has changed from the previous determination result, the determination device 10 reports to the user that the determination result has changed.

Diagnostic information acquired by the record generation function 151 includes information relating to an estimation result of a medicine effect. The diagnostic information is, for example, information indicating “diagnosed that pneumonia improved”, “diagnosed that pneumonia did not improve”, “no determination as to improvement of pneumonia”, etc. regarding a specific medicine used for treatment of pneumonia. For example, in epidemic diseases such as infectious diseases, even if a disease improved in a test result, the situation may be observed for several days. For example, if the improved state of the disease is maintained even after several days have passed since the test result of the improved disease was obtained, the administered medicine is diagnosed as effective for treatment of the subject. On the other hand, if the disease worsens several days after the test result of the improved disease is obtained, it is diagnosed that the administered medicine is not effective for treatment of the subject. A diagnostic result regarding a medicine effect that is once decided at the time of a test (hereinafter, referred to as an initial diagnosis result of a medicine effect) is, for example, recorded as natural language in the item of “estimation of medicine effect” of the medical information system 30. In addition, the initial diagnosis result of a medicine effect is recorded in the item of “estimation of medicine effect” of the record. A result of a confirmed diagnosis regarding a medicine effect is, for example, recorded as natural language in the item of “confirmed diagnosis of medicine effect” of the medical information system 30. In addition, the result of a confirmed diagnosis of a medicine effect is, for example, recorded in the item of “confirmed diagnosis of medicine effect” of the record.

The processing circuitry 15 further receives information relating to a determination subject by the reception function 152. Specifically, the processing circuitry 15 extracts a record of a subject for whom a confirmed diagnosis for a medicine effect is not recorded from all the records stored in the memory 11, and acquires information recorded in the extracted record as information relating to the determination subject. The determination subject is an example of a first subject. The information relating to the determination subject is an example of first subject information.

The processing circuitry 15 outputs a determination result regarding estimation of a medicine effect based on a determination model and information relating to the determination subject by the determination function 153. The processing circuitry 15 outputs the determination result obtained from the determination model to the test database 40, the display 13, a printing device connected to the determination device 10, etc. The determination result is an example of first information. The determination result may be referred to as first risk information.

The processing circuitry 15 acquires information relating to a subject with confirmed diagnosis by the acquisition function 154. In the present embodiment, as the information relating to a subject with confirmed diagnosis, information relating to a subject for whom a confirmed diagnosis regarding a medicine effect is recorded is acquired. Specifically, the processing circuitry 15 extracts a record of a subject with confirmed diagnosis of medicine effect from all the records stored in the memory 11, and acquires the information relating to the subject with confirmed diagnosis of medicine effect from the extracted record. The information relating to the subject with confirmed diagnosis of medicine effect includes history information of the subject with confirmed diagnosis of medicine effect. The history information includes test information and a confirmed diagnosis result. The subject with confirmed diagnosis is an example of a second subject. The information relating to the subject with confirmed diagnosis is an example of second subject information. The confirmed diagnosis result of the subject with confirmed diagnosis is an example of diagnostic information of the second subject.

The processing circuitry 15 updates, by the update function 155, a determination model based on the information relating to the subject with confirmed diagnosis of medicine effect and the diagnostic information of the subject with confirmed diagnosis of medicine effect. At this time, the processing circuitry 15 updates the determination model using the confirmed diagnosis result of the subject with confirmed diagnosis.

A subject with confirmed diagnosis having a confirmed diagnosis of medicine effect is added to the test database 40 daily. The processing circuitry 15 updates, by the update function 155, a determination condition regarding estimation of a medicine effect by performing the above-described classification/totalizing processing on all the subjects with confirmed diagnosis including the newly added subjects with confirmed diagnosis. By updating the determination condition, the determination model is updated.

The processing circuitry 15 outputs a determination result after the update based on an updated determination model and the information relating to the determination subject by the determination function 153. The processing circuitry 15 performs a re-determination using the determination model, updates the determination result based on a re-determination result of the determination model, and outputs the determination result after the update to the test database 40, the display 13, a printing device connected to the determination device 10, etc. The determination result after the update is an example of second information. The determination result after the update may be referred to as second risk information.

The processing circuitry 15 compares the determination results before and after the update for each of the determination subjects, and outputs a comparison result regarding a subject whose determination results before and after the update are different, by the comparison function 156.

Next, an operation of medical determination support processing executed by the determination device 10 will be described. In the present embodiment, the medical determination support processing is processing of collecting test information and confirmed diagnosis results regarding medicine effects for a plurality of subjects, estimating a medicine effect of a specific medicine for a determination subject based on the collected results, performing determination regarding the estimation of the medicine effect, and when a determination result has changed from a previous determination result, outputting information indicating that the determination result has changed.

(Medical Determination Support Processing)

(Step S101)

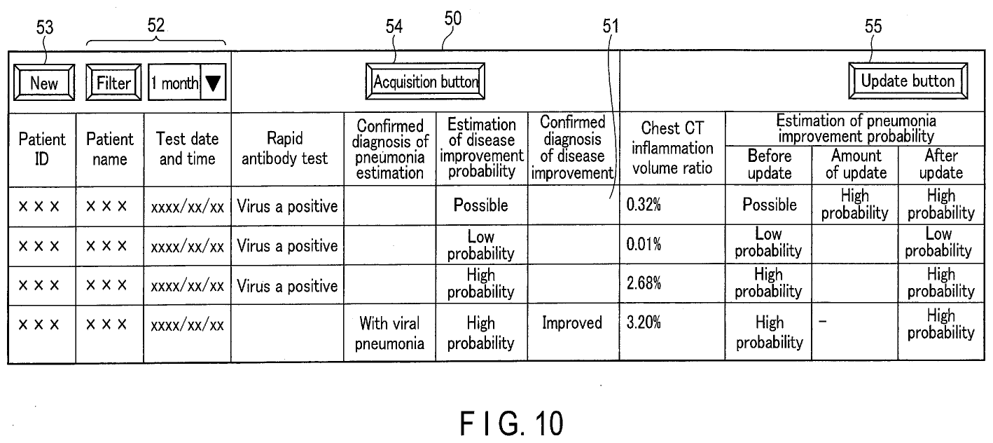

The processing circuitry 15 causes the display 13 to display the data management screen 50 based on the records stored in the memory 11, by the display control function 157. FIG. 10 is a diagram showing an example of the data management screen 50. In the present embodiment, an initial diagnosis result of a medicine effect is further displayed in the display column of the diagnostic information of the data display part 51.

(Step S102)

When an operation is input in the record creation instruction input part 53, the processing circuitry 15 generates a new record by the record generation function 151 in the same manner as in the first embodiment.

(Step S103)

When an operation is input in the data collection instruction input part 54, the processing circuitry 15 acquires electronic medical records of the subjects displayed in the data display part 51 from the medical information system 30, and collects test information and diagnostic information of subjects for whom “pneumonia” is recorded in the item of “suspicious diagnosis” in the same manner as in the first embodiment. As a result, test information and diagnostic information that have not been recorded in the record so far are newly acquired. Only the extracted records are displayed in the data display part 51. In the example of FIG. 10, among the records created in the last one month, there are four subjects for whom “pneumonia” is recorded in the “suspicious diagnosis” item of the electronic medical record.

(Step S104)

When an operation is input in the update instruction input part 55, the processing circuitry 15 extracts a subject whose confirmed diagnosis of medicine effect does not exist from the records displayed in the data display part 51 as a determination subject. Then, the processing circuitry 15 acquires a determination result by executing the determination process in the same manner as in the first embodiment. The determination process of the present embodiment is a process of performing determination regarding estimation of a medicine effect. In the determination process, a determination result using a determination model can be obtained for a subject for whom the determination is performed for the first time. For a subject for whom a second or subsequent determination is performed, a latest determination result can be obtained by using a determination model that reflects a diagnostic result of a subject with confirmed diagnosis added from the date of the previous determination to the time of the determination this time. The determination result obtained by the determination process is stored in the record.

(Step S105)

The processing circuitry 15 compares determination results of the last two times stored in the record of the determination subject by the comparison function 156. When the determination results are different, the processing circuitry 15 extracts a change in determination result and displays it in the data display part 51.

Hereinafter, advantageous effects of the medical information processing system 1 having the determination device 10 according to the present embodiment will be described.

In order to evaluate effectiveness of a newly developed medicine for a specific subject having contracted an infectious disease or a new disease, records of a large number of subjects are newly generated and treatment progress and health conditions of the subjects are recorded daily in the test database 40. In addition, a subject with confirmed diagnosis having a confirmed diagnosis of a medicine effect of the newly developed medicine is added to the test database 40 daily.

Effectiveness of the newly developed medicine may change over a course of a few days as information on subjects having a confirmed diagnosis of a medicine effect is gathered. Moreover, in the case of epidemic diseases, a prediction result of an effect of a therapeutic agent may change rapidly.

The medical information processing system 1 according to the present embodiment also uses test information of target subject as history information to receive subject information based on the history information of the determination subject, and based on a determination model for a specific disease and the subject information of the determination subject, outputs information relating to a probability of the specific disease for the determination subject. In the present embodiment, as the information relating to a probability of a specific disease, a medicine effect of a medicine used as a therapeutic agent for the specific disease is used. The medicine effect is a probability that a condition of a subject to whom a specific medicine has been administered will improve. In addition, as the history information, the test information of the target subject is used.

With the above configuration, according to the medical information processing system 1 according to the present embodiment, a determination model is updated using confirmed diagnosis results of all the subjects with confirmed diagnosis including a subject to whom a confirmed diagnosis of medicine effect is newly added. As a result, a diagnosis result of a subject for whom a confirmed diagnosis of medicine effect is made from the day when the previous determination of a medicine effect was made based on a simple test such as a rapid antibody test to the time when the determination this time is made is reflected in the determination model. Then, by performing determination of a medicine effect of a specific medicine from a result of a simple test such as a rapid antibody test using the updated determination model, it is possible to obtain a determination result that reflects a change in medicine effect prediction of a therapeutic agent.

For example, when there are two types of medicines that may be effective for a specific disease, it is possible to determine which medicine is effective for a subject. As a result, even if the medicine effect prediction changes significantly in a short period of time, a medicine to be administered to the subject can be changed promptly and appropriate measures can be taken for the patient.

Further, the medical information processing system 1 according to the present embodiment compares a determination result after the update and a determination result before the update for each of the determination subjects, and outputs a comparison result for a subject whose determination results before and after the update are different. For example, when the current determination result has changed from the previous determination result, the data management screen 50 displaying the change in determination result is displayed on the display 13. By checking the data management screen 50 on which the change in determination result is displayed, the user can grasp the subject whose medicine effect prediction result has changed and take appropriate measures.

According to at least one embodiment described above, even when a probability for a specific disease changes significantly in a short period of time, a determination result reflecting the changed probability can be output.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical information processing system comprising:
a storage configured to store a determination model that determines a probability for a specific disease; and
processing circuitry configured to:
receive first subject information based on a history of a first subject;
output first information including the probability of the first subject based on the determination model and the first subject information;
acquire second subject information based on a history of a second subject;
update the determination model based on the second subject information; and
output second information including the probability of the first subject based on an updated determination model and the first subject information.

2. The medical information processing system according to claim 1, wherein
the determination model determines a probability that a specific subject has contracted the disease as the probability.

3. The medical information processing system according to claim 2, wherein
the history includes at least one of a behavior history and a medical interview result.

4. The medical information processing system according to claim 2, wherein
the processing circuitry is further configured to compare the first information and the second information for each of a plurality of the first subjects, and output a comparison result for a subject with the first information and the second information being different from each other.

5. The medical information processing system according to claim 2, wherein
the second subject information includes a confirmed diagnosis of the second subject regarding the disease, and
the processing circuitry is further configured to update the determination model based on the confirmed diagnosis of the second subject.

6. The medical information processing system according to claim 5, wherein
the second subject information includes a test result and a diagnostic finding of the second subject, and
the processing circuitry is further configured to generate the confirmed diagnosis of the second subject based on the test result and the diagnostic finding of the second subject.

7. The medical information processing system according to claim 5, wherein
the processing circuitry is further configured to extract a behavior item having a certain amount or more of influence on a diagnostic result based on at least one of a behavior history of the second subject and a medical interview result of the second subject, and the confirmed diagnosis of the second subject.

8. The medical information processing system according to claim 7, wherein
the processing circuitry is further configured to add the extracted behavior item to a question item of a medical interview sheet.

9. The medical information processing system according to claim 2, wherein the processing circuitry is further configured to:
update the determination model by calculating a disease probability of each of a plurality of clusters, and classifying the second subject into the clusters so that a difference in the disease probability between the clusters becomes large; and
determine in which one of the clusters the first subject is included, and output the second information based on a disease probability of a cluster in which the first subject is included.

10. The medical information processing system according to claim 9, wherein the processing circuitry is further configured to:
acquire a behavior date and time of the second subject based on at least one of a behavior history and a medical interview result of the second subject;
classify the second subject into the clusters based on the behavior date and time of the second subject; and
output the second information based on a behavior date and time of the first subject.

11. The medical information processing system according to claim 1, wherein
the determination model determines a probability that a condition of a subject to whom a specific medicine is administered will improve as the probability.

12. The medical information processing system according to claim 11, wherein
the history includes test information of the subject.

13. The medical information processing system according to claim 11, wherein
the processing circuitry is further configured to compare the first information and the second information for each of a plurality of the first subjects, and output a comparison result for a subject with the first information and the second information being different from each other.

14. The medical information processing system according to claim 11, wherein
the second subject information includes a confirmed diagnosis of the second subject regarding an effect of the medicine, and
the processing circuitry is further configured to update the determination model based on the confirmed diagnosis of the second subject.

15. A medical information processing method comprising:
receiving first subject information based on a history of a first subject;
based on a determination model that determines a probability for a specific disease and the first subject information, outputting first information including a probability of the first subject regarding the disease;
acquiring second subject information based on a history of a second subject;
updating the determination model based on the second subject information; and
outputting second information including the probability of the first subject based on an updated determination model and the first subject information.
